(19) United States
(12) Patent Application Publication
(10) Pub. No.: US 2019/0133495 A1
(43) Pub. Date: May 9, 2019

(54) SYSTEMS AND METHODS FOR IMPROVING ATHLETIC TRAINING, PERFORMANCE AND REHABILITATION

(71) Applicant: Duke University, Durham, NC (US)

(72) Inventors: Brian P. Mann, Durham, NC (US); Dane Sequeira, Durham, NC (US); James Turner, Durham, NC (US); Jared Little, Durham, NC (US); Michael Mazzoleni, Morrisville, NC (US)

(21) Appl. No.: 16/096,854

(22) PCT Filed: Apr. 26, 2017
(86) PCT No.: PCT/US17/29709

§ 371 Date: Oct. 26, 2018

Related U.S. Application Data
(60) Provisional application No. 62/328,207, filed on Apr. 27, 2016.

(51) Int. Cl. A61B 5/11; G09B 19/00; A61B 5/00

(52) U.S. Cl. CPC A61B 5/1118; G09B 19/0038; A61B 5/6898; A61B 5/14542; A61B 5/681; A61B 5/1121; A61B 5/6803; A61B 5/7275

(57) ABSTRACT

Systems and methods for improving athletic training, performance and rehabilitation. In one example, the system and method perform or include receiving, with an electronic processor, data associated with the subject; generating, with a parameter estimation algorithm, a parameter value for each of a plurality of parameters associated the subject; determining, with the electronic processor, an effect of training on a performance variable, p, associated with the subject; and determining an exercise routine based on optimizing the performance variable.

### FIELD

Embodiments described herein relate to systems and methods for improving athletic training, performance and rehabilitation.

### BACKGROUND

Training is widely accepted as a method to improve an individual's performance in sports. However, athletes have typically relied on experience, heuristics, and rough approximations to design their training routines. Understanding the relationship between training, physiological adaptations, and performance is one of the primary goals for athletes, coaches, and physicians. Despite many advances in science related to physiological insights, sensor technology, and computational methods, the primary methods for designing athletic training programs remain based on “tribal knowledge” or traditional wisdom.

### SUMMARY

Due to, among other things, deficiencies in current techniques, an improved model for prediction of athletic performance and designing optimal training strategies is desired.

The present disclosure provides, in part, nonlinear performance prediction models that account for nonlinear aspects of physiological adaptation and methods incorporating said performance prediction models to better quantify and improve athletic performance and rehabilitation.

Embodiments provided herein utilize, among other things, nonlinear mathematical models and a heuristic algorithm to predict and improve training routines for individuals participating in a variety of exercise activities, including but not limited to running, biking, swimming, strength training, rowing, multi-sport training (such as triathlon), and others.

Some embodiments provide a method for determining the optimal exercise routine of a subject, the method comprising, consisting of, or consisting essentially of: (a) receiving, at an electronic processor, information relating to the subject; (b) calibrating the model using a heuristic parameter estimation algorithm; (c) calculating, using the electronic processor, the effect of training on performance according to the equations:

$$p = {p}_{o} \pm ( f - u )$$

$$\dot{f} + \frac{1}{{τ}_{1}} {f}^{α} = {k}_{1} σ ( t )$$

$$\dot{u} + \frac{1}{{τ}_{2}} {u}^{β} = {k}_{2} σ ( t )$$

where σ(t) is the training stress impulse, τ₁, τ₂, α, β, k₁, and k₂ are subject-specific constants that represent the physiological attributes of the subject, p₀ is the baseline performance output of the subject in an untrained state, f is the fitness of the subject, u is the fatigue of the subject, and p is the performance of the subject; and (d) determining the optimal exercise routine based on the results of a heuristic optimization algorithm.

Another aspect of the present disclosure provides a method for predicting the performance of a subject comprising, consisting of, or consisting essentially of: (a) receiving, at an electronic processor, information relating to the subject; (b) calibrating the model using a heuristic parameter estimation algorithm; (c) calculating, using the electronic processor, the effect of training on performance according to the equations:

$$p = {p}_{o} \pm ( f - u )$$

$$\dot{f} + \frac{1}{{τ}_{1}} {f}^{α} = {k}_{1} σ ( t )$$

$$\dot{u} + \frac{1}{{τ}_{2}} {u}^{β} = {k}_{2} σ ( t )$$

where σ(t) is the training stress impulse, τ₂, α, β, k₁, and k₂ are subject-specific constants that represent the physiological attributes of the subject, p₀ is the baseline performance output of the subject in an untrained state, f is the fitness of the subject, u is the fatigue of the subject, and p is the performance of the subject; and (d) determining the predicted performance of the subject based on the results of the calculation, with uncertainty bounds on the performance predictions.

Another aspect of the present disclosure provides a method for continually improving or optimizing the training routine of a subject comprising, consisting of, or consisting essentially of: (a) receiving at the processor information relating to the subject; (b) calibrating the model using a heuristic parameter estimation algorithm; (c) calculating using the processor, the effect of training on performance according to the equations:

$$p = {p}_{o} \pm ( f - u )$$

$$\dot{f} + \frac{1}{{τ}_{1}} {f}^{α} = {k}_{1} σ ( t )$$

$$\dot{u} + \frac{1}{{τ}_{2}} {u}^{β} = {k}_{2} σ ( t )$$

where σ(t) is the training stress impulse, τ₁, τ₂, α, β, k₁, and k₂ are subject-specific constants that represent the physiological attributes of the subject, p is the baseline performance output of the subject in an untrained state, f is the fitness of the subject, u is the fatigue of the subject, and p is the performance of the subject; (d) determining the optimal exercise routine based on the results of a heuristic optimization algorithm; (e) repeating steps (a)-(c), in which new information is added in step (a); and (f) re-determining the optimal exercise routine based on the results of a heuristic optimization algorithm.

In some embodiments, the heuristic parameter estimation algorithm comprises a genetic algorithm. Other parameter estimation algorithms may also be employed. In some embodiments, the heuristic algorithm for training optimization comprises a genetic algorithm. Other optimization may also be employed.

In another embodiment, the information is selected from the group consisting of (i) physiological attributes of the subject; (ii) calibration data consisting of measured training stress and performance data from past exercise; (iii) training stress; (iv) fitness and (v) fatigue and combinations thereof.

Some embodiments provide a system for determining the optimal exercise routine of a subject comprising, consisting of; or consisting essentially of: an interface for inputting information relating to the subject; an electronic processor; and a memory storage device for storing the information relating to the subject and instructions executable by the electronic processor for computing an optimal exercise routine, the instructions comprising, consisting of, or consisting essentially of: (a) receiving at the electronic processor information relating to any of the following: (i) physiological attributes of the subject; (ii) calibration data consisting of training and performance data from past exercise; (iii) training stress; (iv) fitness and (v) fatigue; (b) calibrating the model using a heuristic parameter estimation algorithm; (c) calculating, using the electronic processor, the effect of training on performance according to the equations:

$$p = {p}_{o} \pm ( f - u )$$

$$\dot{f} + \frac{1}{{τ}_{1}} {f}^{α} = {k}_{1} σ ( t )$$

$$\dot{u} + \frac{1}{{τ}_{2}} {u}^{β} = {k}_{2} σ ( t )$$

where σ(t) is the training stress impulse, τ₁, τ₂, α, β, k₁, and k₂ are subject-specific constants that represent the physiological attributes of the subject, p₀ is the baseline performance output of the subject in an untrained state, f is the fitness of the subject, u is the fatigue of the subject, and p is the performance of the subject; and (d) determining the optimal exercise routine based on the results of a heuristic optimization algorithm.

Some embodiments provide a system for predicting the performance of a subject comprising, consisting of, or consisting essentially of: an interface for inputting information relating to the subject; an electronic processor; and a memory storage device for storing the information relating to the subject and instructions executable by the electronic processor for computing the predicted performance of the subject, the instructions comprising, consisting of, or consisting essentially of: (a) receiving at the electronic processor information relating to any of the following: (i) physiological attributes of the subject; (ii) calibration data consisting of measured training stress and performance data from past exercise; (iii) training stress; (iv) fitness and (v) fatigue; (b) calibrating the model using a heuristic parameter estimation algorithm; (c) calculating using the electronic processor, the effect of training on performance according to the equations:

$$p = {p}_{o} \pm ( f - u )$$

$$\dot{f} + \frac{1}{{τ}_{1}} {f}^{α} = {k}_{1} σ ( t )$$

$$\dot{u} + \frac{1}{{τ}_{2}} {u}^{β} = {k}_{2} σ ( t )$$

where σ(t) is the training stress impulse, τ₁, τ₂, α, β, k₁, and k₂ are subject-specific constants that represent the physiological attributes of the subject, p₀ is the baseline performance output of the subject in an untrained state, f is the fitness of the subject, u is the fatigue of the subject, and p is the performance of the subject; and (d) determining the predicted performance of the subject based on the results of the calculation, with uncertainty bounds on the performance predictions.

Some embodiments provides a system for continually improving the optimal training routine of a subject comprising, consisting of, or consisting essentially of: an interface for inputting information relating to the subject; an electronic processor; and a memory storage device for storing the information relating to the subject and instructions executable by the electronic processor for computing the optimized training routine of the subject, the instructions comprising, consisting of, or consisting essentially of: (a) receiving at the electronic processor information relating to any of the following: (i) physiological attributes of the subject; (ii) calibration data consisting of measured training and performance data from past exercise; (iii) training stress; (iv) fitness, and (v) fatigue; (b) calibrating the model using a heuristic parameter estimation algorithm; (c) calculating, using the electronic processor, the effect of training on performance according to the equations:

$$p = {p}_{o} \pm ( f - u )$$

$$\dot{f} + \frac{1}{{τ}_{1}} {f}^{α} = {k}_{1} σ ( t )$$

$$\dot{u} + \frac{1}{{τ}_{2}} {u}^{β} = {k}_{2} σ ( t )$$

where σ(t) is the training stress impulse, τ₁, τ₂, α, β, k₁, and k₂ are subject-specific constants that represent the physiological attributes of the subject, p₀ is the baseline performance output of the subject in an untrained state, f is the fitness of the subject, u is the fatigue of the subject, and p is the performance of the subject; and (d) determining the optimal exercise routine based on the results of a heuristic optimization algorithm; (e) repeating steps (a)-(c), in which new information is added in step (a); and (f) re-determining the optimal exercise routine based on the results of a heuristic optimization algorithm.

Other aspects of the invention will become apparent by consideration of the detailed description and accompanying drawings.

### BRIEF DESCRIPTION OF THE DRAWINGS

The accompanying figures, where like reference numerals refer to identical or functionally similar elements throughout the separate view, together with the detailed description below, are incorporated in and form part of the specification, and serve to further illustrate embodiments of concepts that include the claimed embodiments, and explain various principles and advantages of those embodiments.

FIG. 1 illustrates a block diagram of a system for improving athletic training, performance and rehabilitation, in accordance with some embodiments.

FIG. 2 illustrates various software programs in the memory shown in FIG. 1, in accordance with some embodiments.

FIG. 3 is a table showing several parameter values and initial conditions for simulations and examples, in accordance with some embodiments.

FIG. 4 is a plot illustrating how the steady-state performance changes with training stress, in accordance with some embodiments.

FIG. 5 is a plot illustrating the compounding effects of steady-state fitness and fatigue with increases in training stress, in accordance with some embodiments.

FIG. 6 is a plot illustrating how athlete performance changes with time, in accordance with some embodiments.

FIG. 7 is a plot illustrating (a) performance and (b) daily training stress changes for a 7-day taper, in accordance with some embodiments.

FIG. 8 is a plot illustrating (a) final performance and (b) constant daily training stress for various taper lengths for two cases: (i) the constant daily training stress σ=σ₁ (solid line); and (ii) the constant daily training stress that produces the highest performance at the end of 84 days for a given taper length (dashed line), in accordance with some embodiments.

FIG. 9 is a plot illustrating (a) performance, and (b) prescribed daily training stress for a maximum daily training stress constraint σ_{allow}=300, in accordance with some embodiments.

FIG. 10 is a plot illustrating (a) performance, (b) acute training load (ATL), and (c) daily training stress over time wherein the optimization is constrained by σ_{allow}=300 and ATL_{allow}=200, in accordance with some embodiments.

FIG. 11 is a plot illustrating (a) performance, and (b) training stress when a person-specific fatigue constraint and a daily maximum training stress of σ_{allow}=300 are applied, in accordance with some embodiments.

FIG. 12 is a plot illustrating (a) performance, (b) ATL/CTL ratio, and (c) daily training stress as optimized using the ATL/CTL constraint, in accordance with some embodiments.

FIG. 13 is a plot illustrating (a) performance, (b) u/f, and (c) daily training stress as optimized using the u/f constraint. A daily maximum stress of (τ_{allow}=300 and (u/f)_{allow}=0.8 are imposed as constraints, in accordance with some embodiments.

FIG. 14 is a plot illustrating (a) performance, and (b) daily training stress when a person-specific f-based constraint and a daily maximum training stress of σ_{allow}=300 are applied, in accordance with some embodiments.

FIG. 15 is a plot illustrating (a) performance, (b) u/f, and (c) daily training stress when using the u-based, f-based, and u/f-based constraints, in accordance with some embodiments.

FIG. 16 is a plot illustrating (a) the performance data and the predicted performance values using the parameter set found by a genetic algorithm, and (b) the experimental training stress measurements, in accordance with some embodiments.

FIG. 17 illustrates a normalized 2-D histogram of predicted performance using parameters for randomly selected sets of trials where the counts are normalized such that the sum in each vertical slice is 1 and experimental data points are overlaid as dots, in accordance with some embodiments.

FIG. 18 illustrates density estimation of final performance for randomly selected sets of trials where the vertical axis was normalized such that the total area under the histogram was 1, in accordance with some embodiments.

FIG. 19 is an example of a training routine for a subject, in accordance with some embodiments.

FIG. 20 is a flow chart of a method for predicting performance of an athlete, in accordance with some embodiments.

FIG. 21 is a flow chart of a method for generating an optimal training schedule and predicted performance, in accordance with some embodiments.

FIG. 22 is a flow chart of a method determining an optimal exercise routine of a subject, in accordance with some embodiments.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments provided herein. The system and method components have been represented where appropriate by conventional symbols in the drawings, showing only those specific details that are pertinent to understanding the embodiments so as not to obscure the disclosure with details that will be readily apparent to those of ordinary skill in the art having the benefit of the description herein.

### DETAILED DESCRIPTION

For the purposes of promoting an understanding of the principles of the present disclosure, reference will now be made to one or more embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates. Furthermore, other embodiments may exist that are not described herein. Also, the functionality described herein as being performed by a single component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. It should not also be noted that a plurality of hardware and software based devices may be utilized to implement various embodiments.

Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used in the present application, “non-transitory computer-readable medium” comprises all computer-readable media but does not consist of transitory, propagating signals. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

Articles “a” and “an” are used herein to refer to one or to more than one (i.e. at least one) of the grammatical object of the article. By way of example, “an element” means at least one element and can include more than one element. In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual relationship or order between such entities or actions. Unless otherwise defined, all technical terms used herein have the same meaning as commonly understood by one of ordinary skill in the art to which this disclosure belongs.

Prior studies investigating the use of dynamical systems models to predict athletic performance based on training have been linear in nature and fail to capture several important nonlinear physiological features such as performance saturation and over-training, which makes it nearly impossible to gain long-term athletic insights or design optimal training strategies using these models. Embodiments provided herein describe the design and implementation of models that take into account these nonlinear physiological features. Systems and methods provided herein allow for the capture of a diminishing rate of return or performance saturation and over training. Some embodiments provide for designing optimal training strategies based on the personal physiology, constraints, and performance goals of an individual.

Embodiments provided herein use the term “performance” to represent a broad sense of physical activity including various sports and progression in rehabilitation. In some embodiments, performance measurements include time, distance, pace, etc. In some embodiments, performance can be defined in the context of physical rehabilitation. For example, a performance measurement may include the weight an individual can lift, a level of balance for the individual, the quality of movement and/or biomechanics of an individual, etc. Embodiments provided herein may use the same approach used to obtain an optimal exercise plan to help an individual needing to undergo physical rehabilitation.

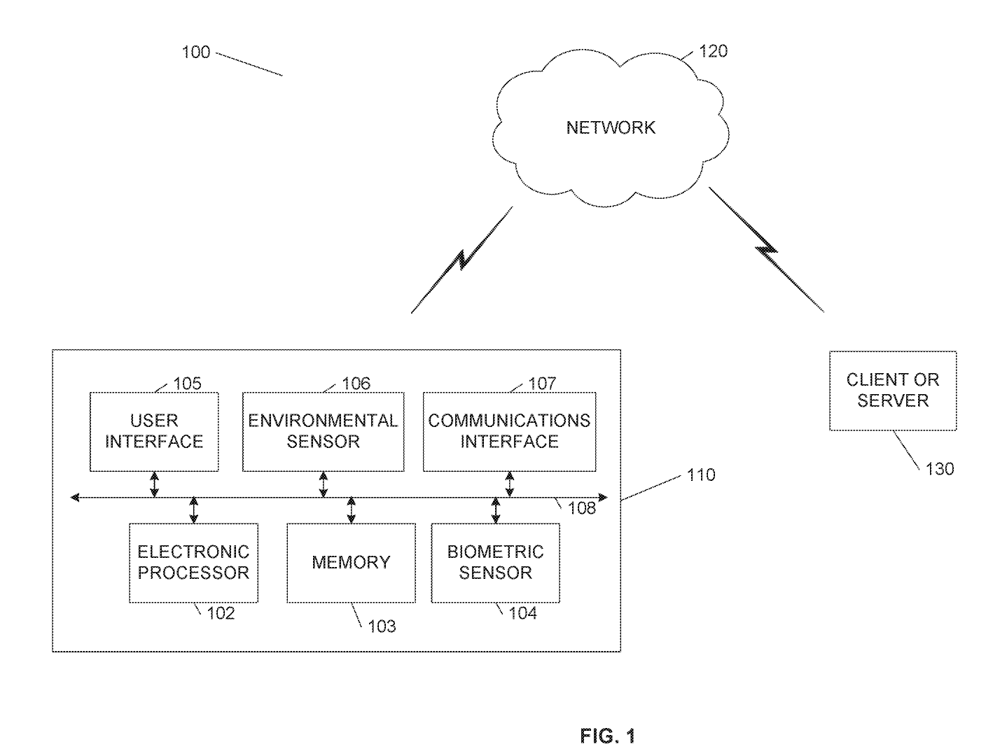

FIG. 1 illustrates a block diagram of a system 100 for optimizing athletic training, performance and rehabilitation, in accordance with some embodiments. System 100 includes a computing device 110, a network 120, and a server 130. In some embodiments, the computing device 110 and/or the server 130 may combine hardware, software, firmware, and system on-a-chip technology to implement the methods provided herein. In some embodiments, the computing device 110 includes an electronic processor 102, a memory 103, a biometric sensor 104, a user interface 105, an environmental sensor 106, a communication interface 107, and a bus 108. In some embodiments, the server 130 may include the memory 103 and one or more components (the user interface 105, electronic processor 102, communication interface 107, etc.) included in the computing device 110. The computing device 110 may comprise any device capable of processing instructions and transmitting data to and from the subject, including portable communication devices such as wireless phones, personal digital assistants, palm computers, laptop computers, wearable devices, such as smart glasses, smart watches, headphones, and the like.

In one embodiment, the electronic processor 102 may include at least one microprocessor and be in communication with at least one microprocessor. The microprocessor interprets and executes a set of instructions stored in the memory 103. The one or more software programs within memory 103 may be configured to implement the methods described herein. In some embodiments, the memory 103 includes, for example, random access memory (RAM), read-only memory (ROM), and combinations thereof. In some embodiments, the memory 103 has a distributed architecture, where various components are situated remotely from one another, but may be accessed by the electronic processor 102. The instructions may comprise any set of instructions to be executed directly (such as machine code) or indirectly (such as scripts) by the processor.

Data may be retrieved, stored, entered (for example, by the subject) or modified by the electronic processor in accordance with the instructions. The data may be stored as a collection of data. Although embodiments are not limited by any particular data structure, the data may be stored in memory, in a relational database as a table having a plurality of different fields and records, or as an XML file. The data may also be formatted in any computer readable format such as, but not limited to, binary values, ASCII or EBCDIC (Extended Binary-Coded Decimal Interchange Code). Moreover, any information sufficient to identify the relevant data may be stored, such as descriptive text, proprietary codes, pointers, or information which is used by a function to calculate the relevant data.

The biometric sensor 104 may include one or more sensors capable of measuring various physiological parameters associated with a subject (for example, an athlete). In some embodiments, the sensor 104 includes a device capable of measuring physiological information such as a heart rate, an oxygen volume (VO₂), oxygen saturation (SmO₂), blood lactate level (BLa), etc. of the subject. In some embodiments, the sensor 104 includes one or more of a velocity sensor, a torque sensor, a force sensor, a velocity sensor, a power meter, a GPS system, a timing device, an inertial measurement unit (IMU), an accelerometer, a gyroscope, a magnetometer, a step sensor, a position sensor.

The user interface 105 provides a mechanism for a user to interact with the computing device 110. The user interface 105 may include input devices such as a display, keys, buttons, touch-pad or touch-screen device, and other related devices. In some embodiments, the user interface 105 may also interact with or be controlled by software programs including speech-to-text and text-to-speech interfaces. In some embodiments, the user interface 105 includes a command language interface, for example, a software-generated command language interface that includes elements configured to accept user inputs, for example, program-specific instructions or data. In some embodiments, the software-generated components of the user interface 105 include menus that a user may use to choose particular commands from lists displayed on a display. In some embodiments, the display and/or the input/output port may provide a graphical user interface (GUI) for the user to enter and receive information. For example, the display may depict a series of prompts requesting information from the user. In response to these prompts, the user may enter data by, for example, selecting an item from a drop-down menu, entering information in predefined data fields, or linking information from a separate application or device.

The environmental sensor 106 may include one or more sensors capable of measuring environmental aspects such as temperature, wind speed, humidity, etc.

The communication interface 107 provides the computing device 110 a communication gateway with an external network (for example, a wireless network, the internet, etc.) The communication interface 107 may include, for example, an Ethernet card or adapter or a wireless local area (WLAN) integrated circuit, card or adapter (for example, IEEE standard 802.11 a/b/g/n). The communication interface 107 may include address, control, and/or data connections to enable appropriate communications with the external network.

The bus 108, or other component interconnection, provides one or more communication links among the components of the computing device 110. The bus 108 may be, for example, one or more buses or other wired or wireless connections. The bus 108 may have additional elements, which are omitted for simplicity, such as controllers, buffers (for example, caches), drivers, repeaters, and receivers, or other similar components, to enable communications. The bus 108 may also include address, control, data connections, or a combination of the foregoing to enable appropriate communications among the aforementioned components.

In some embodiments, the electronic processor 102, the biometric sensor 104, the environmental sensor 106, and the memory 103 are included in a single computing device (for example, within a common housing), such as a smart watch, other wearable device or another suitable computing device. In these embodiments, the electronic processor 102 executes a software program that is locally stored in the memory 103 of the computing device 110 to perform the methods described herein. For example, the electronic processor 102 may execute the software program or access and process data (for example, physiological data, training data, etc.) stored in the memory 103. Alternatively or in addition, the electronic processor 102 may execute the software application to access data stored external to the computing device (for example, on a server 130 accessible over a communication network 120 such as the internet). The electronic processor 102 may output the results of processing the accessed data to a display that may be included in the user interface 105 of the computing device 110.

In other embodiments, the electronic processor 102, the biometric sensor 104, the environmental sensor 106, and the memory 103, or a combination thereof may be included in one or more separate devices. For example, in some embodiments, the biometric sensor 104 and/or environmental sensor 106 may be included in a wearable device configured to transmit physiological data and/or environmental data to a server 130 including the memory 103 and one or more components illustrated in FIG. 1 over a wired or wireless communication network or connection. In this configuration, the electronic processor 102 may be included in the server 130 or another device that communicates with the server 130 over a wired or wireless network or connection.

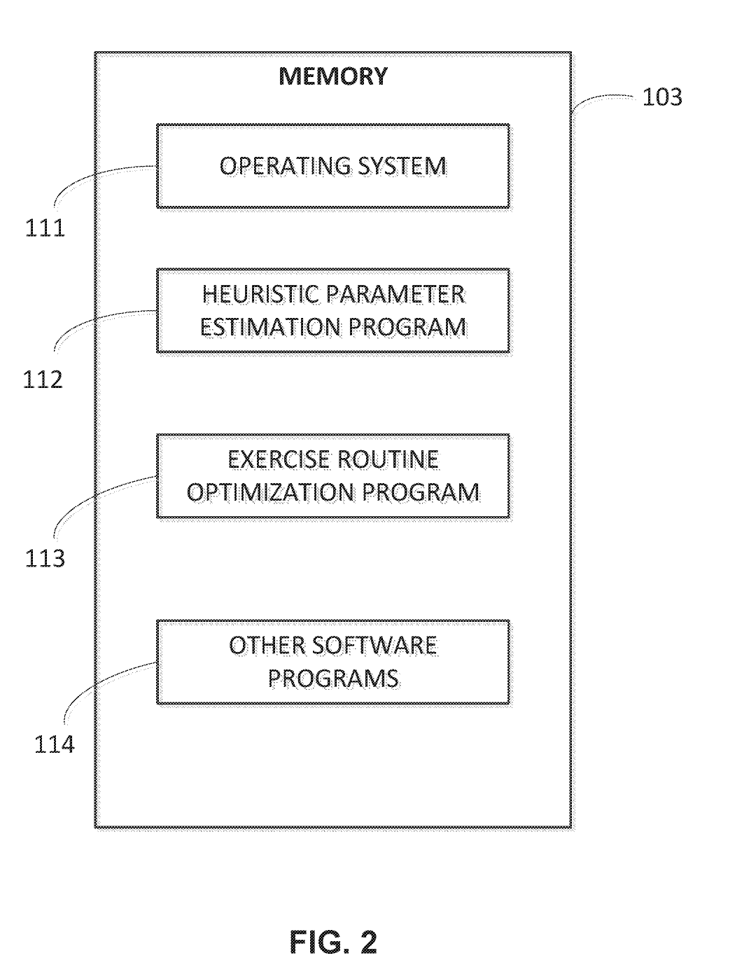

FIG. 2 illustrates various software programs in the memory 103 shown in FIG. 1, in accordance with some embodiments. In some embodiments, the memory 103 includes an operating system 111, a heuristic parameter estimation program 112, an exercise routine optimization program 113, and other software programs 114.

A novel nonlinear performance model is provided herein to improve or optimize training strategies for achieving performance goals under various realistic constraints and also to describe an experimental testing strategy. The nonlinear model incorporates both the positive effects, sometimes called fitness f, and negative effects, sometimes called fatigue u, of training on performance. While changes in both fitness and fatigue can be viewed as the sum of various muscular, psychological, and nutritional factors, these models consider changes in fitness and fatigue due only to training. Performance can then be determined from these values as the difference between fitness and fatigue, p=p₀+f−u, where p₀ is an individual's performance in an untrained state. While short-term performance can often be predicted from this approach, it is impossible to find the training stress that results in an optimal equilibrium performance. Predictions made using this linear modeling approach indicate that equilibrium performance indefinitely improves with increases in training stress, i.e., saturation and the negative effect of over-training are not captured.

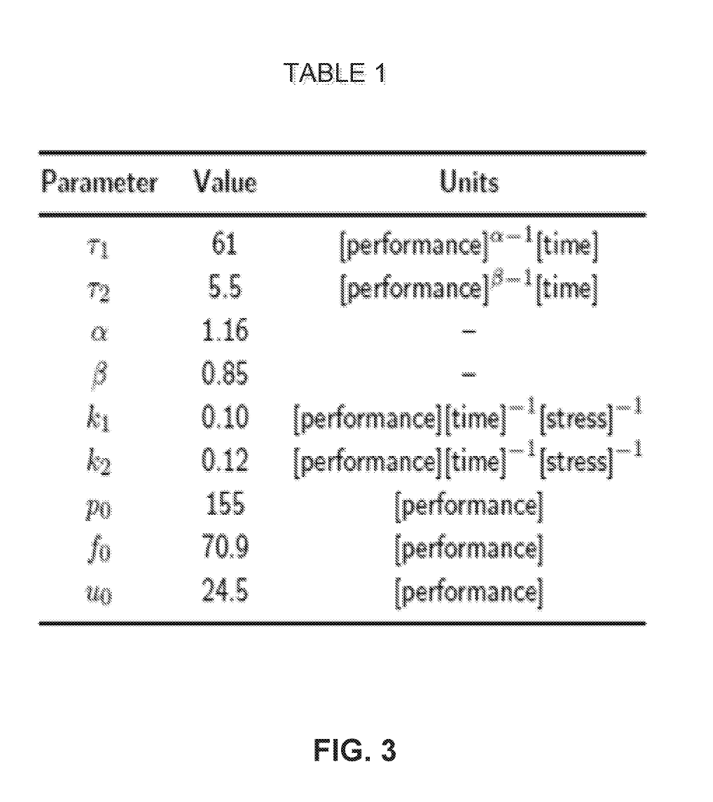

The linear model provided above can be modified to the following set of nonlinear differential equations to capture the effect of training on performance

$$\begin{matrix}\dot{f} + \frac{1}{{τ}_{1}} {f}^{α} = {k}_{1} σ ( t ) & ( 1 ) \\ \dot{u} + \frac{1}{{τ}_{2}} {u}^{β} = {k}_{2} σ ( t ) & ( 2 )\end{matrix}$$

where f is fitness as a function of time, u is fatigue as a function of time, σ is the training stress impulse as a function of time, τ₁ and τ₂ are time constants, k₁ and k₂ are gain terms, α and β are exponents that represent the model's nonlinearities, and t is time. An overdot indicates a time derivative. The parameters are person-specific constants that depend on various physiological factors and can be determined from performance tests and parameter estimation algorithms. The introduction of nonlinearity enables additional phenomena, such as saturation and over-training, to be captured while still accounting for increases and decreases in performance due to training. FIG. 3 is a table showing various parameter values and initial conditions for simulations and examples, in accordance with some embodiments.

Constant Daily Training Stress

The special case of a time invariant or constant daily training stress is useful conceptually to illustrate how Equations (1) and (2) capture the effects of training saturation and over-training. For the results that follow, the person-specific parameters given in the table shown in FIG. 3 are used.

If a constant daily training stress is applied, performance will eventually stagnate or plateau. This case can be explored from the equilibria of Equations (1) and (2) when \dot{f}=\dot{u}=0. The steady-state performance \tilde{p} can then be obtained analytically and becomes

$$\tilde{p} = {p}_{0} \pm [ {( {k}_{1} {τ}_{1} σ )}^{\frac{1}{α}} - {( {k}_{2} {τ}_{2} σ )}^{\frac{1}{β}} ] \qquad ( 3 )$$

The constant daily stress σ₁ that maximizes performance is found by setting d\tilde{p}/dσ=0, which gives

$${σ}_{1} = {[ \begin{matrix}{( \frac{β}{α} )}^{αβ} & \frac{{( {k}_{1} {τ}_{1} )}^{β}}{{( {k}_{2} {τ}_{2} )}^{α}}\end{matrix} ]}^{\frac{1}{α - β}} \qquad ( 4 )$$

Although consistently training above σ₁ will still result in some improvements in performance, the performance increases will be less than the optimal value. Another important stress value is the training stress σ₂ that would result in no performance improvement

$${σ}_{2} = {[ \frac{{( {k}_{2} {τ}_{2} )}^{α}}{{( {k}_{1} {τ}_{1} )}^{β}} ]}^{\frac{1}{β - α}} \qquad ( 5 )$$

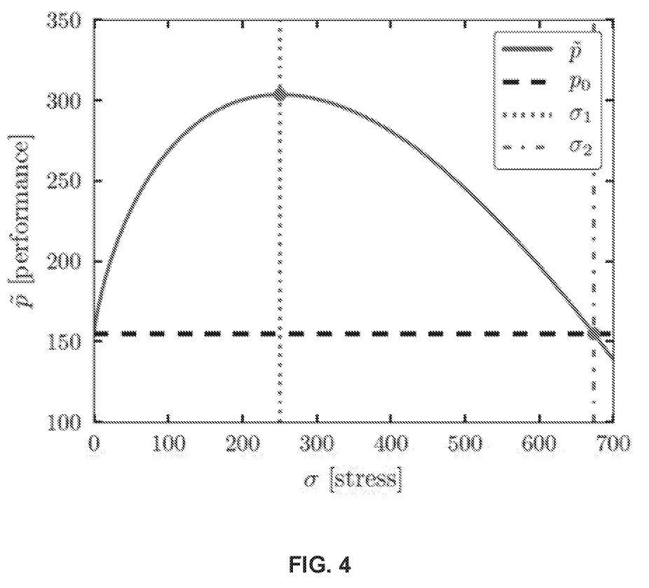

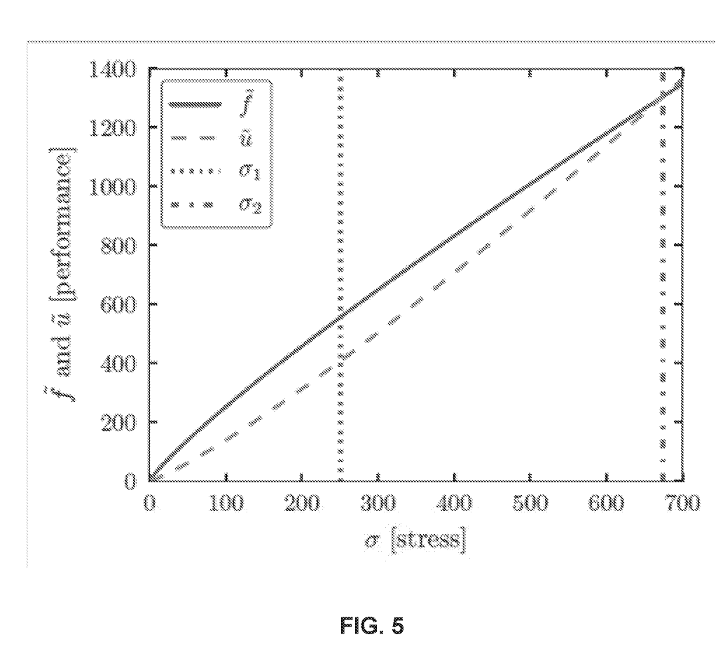

The relationship between σ₁ and σ₂ is shown in FIG. 4 and FIG. 5, which illustrate how these values are related to performance saturation and over-training. FIG. 4 illustrates how the nonlinear performance model captures the physiological phenomena of saturation and over-training. For this plot, the special case of a constant daily training stress is considered. FIG. 5 depicts the compounding effects of fatigue and diminishing returns of fitness gains with increases in training stress. Maximum performance is achieved when \tilde{p}==p₀+\tilde{f}−ũ is maximized, and no performance gains are achieved when \tilde{f}=ũ.

The steady-state trends for fitness, fatigue, and performance are shown in FIG. 4 and FIG. 5. These plots show several interesting aspects. First, there are diminishing returns in performance as training stress is increased. Performance continues to increase along with increases in training stress until σ₁ is reached. If training stress is increased beyond σ₁, then over-training occurs and performance deteriorates as more training stress will have a negative impact on training performance.

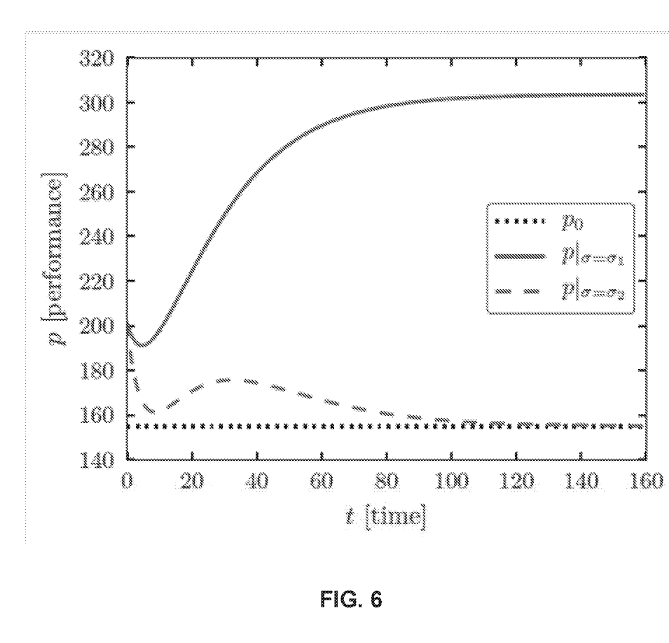

The results shown in FIG. 4 and FIG. 5 only provide the steady-state training and performance relationships. However, FIG. 6 compares the temporal evolution of performance for the constant daily training stresses of σ₁ and σ₂. FIG. 6 illustrates both the super-compensation effect and a plateau in performance after a sufficient period of time.

The analysis presented in this section has only considered the case of a constant daily training stress to provide a simple example that highlights the ability of the nonlinear performance model to capture saturation effects and over-training. However, the more general case, where σ is allowed to vary with time is more realistic. Allowing σ to vary as a function of time more accurately describes practical scenarios in which an individual's training schedule changes on a daily basis. Furthermore, since the nonlinear performance model captures realistic phenomena, such as saturation and over-training, the model can be used to predict performance and design an optimal training routine. Thus, the next section considers different scenarios where σ(t) can be varied to optimize performance for athletes based on their personal physiological characteristics, constraints, and performance goals.

Constrained Optimized Training

The constant daily training stress that was investigated above is useful for understanding the model conceptually, but may not be optimal for use as a training tool. The typical training strategy that athletes employ to prepare for an event is a period of base training with progressively increasing training intensity, followed by a build-up phase at relatively high intensity, and then finishing with a taper phase to reduce fatigue on the competition date. This strategy helps the individual to increase their fitness gradually to avoid injury while maximizing performance on race day.

Depending on an individual's personal physiological characteristics, constraints, and performance goals, the optimal training program can look very different. To explore how the nonlinear model can be applied to optimize a training program and to illustrate the influence of various constraints, this section explores a specific scenario: the case of optimizing performance for a cycling race on the 85^{th} day following twelve consecutive weeks of training. The parameter values that were used during the optimization process are provided in the table shown in FIG. 3. Although the units are arbitrary, the parameter values were chosen to be reasonable for units of days for time, stress, and a cyclist's maximum possible average power output in Watts over a 10-minute interval for performance. In some embodiments, the optimization is performed using genetic algorithms, which are heuristic optimization algorithms based on the idea of natural selection. Genetic algorithms are optimization methods to solve nonlinear problems. In some embodiments, these genetic algorithms are run multiple times for each case studied to ensure consistent results.

Constant Daily Training Stress Constraint with Tapering

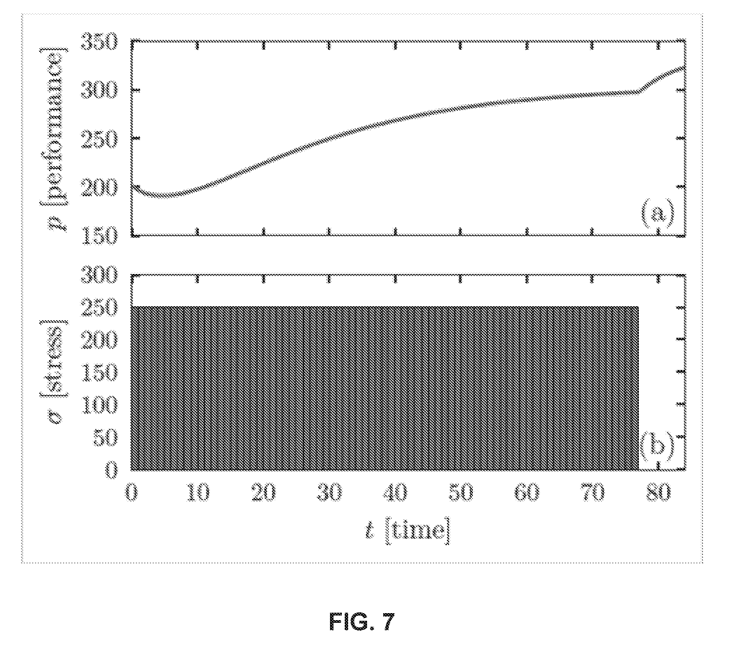

One of the simplest improvements to the constant daily training stress routine described above is to allow a short rest period prior to race day. This period of rest or limited exercise is typically referred to as a taper and allows for the dissipation of accumulated fatigue, thus improving final performance. The considerably shorter time constant of the fatigue τ₂, relative to that of the fitness τ₁, results in improved performance after short rest periods where quick recovery from fatigue outweighs slower losses in fitness. The simplest taper scheme is to stop training a few days before the end of the season. FIG. 7 illustrates an example of this approach, with a notable increase in performance due to tapering. FIG. 7 is a plot showing (a) performance and (b) daily training stress for a 7-day taper. The benefit of the taper can be observed by examining the considerable increase in performance following the onset of the taper.

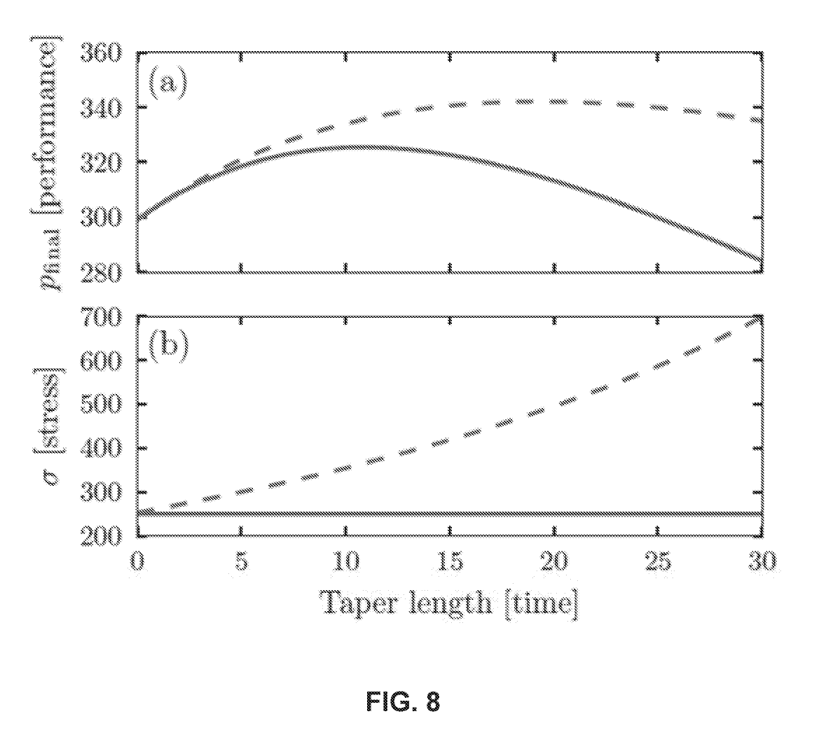

To compare the results with the constant daily stress routine outlined above, two scenarios may be considered: 1) using the value of σ₁ found in Equation (4); and 2) using the stress that produced the highest performance at the end of the season for each taper length. The results of these scenarios can be seen in FIG. 8. FIG. 8 is a plot showing (a) final performance and (b) constant daily training stress for various taper lengths for two cases: 1) the constant daily training stress σ₁ calculated by Equation (4) (solid line); and 2) the constant daily training stress that produces the highest performance at the end of the season for a given taper length (dashed line)

From these results, it is clear that the constant daily training stress scenario is improved by simply implementing a taper. Increasing the length of this taper can improve performance by reducing fatigue, up until the point where the rate of fitness atrophy begins to outweigh the rate of fatigue loss. In the provided example, this optimal taper length is roughly 10 days, as shown by the peak in FIG. 8(a). Additionally, with regard to the case where constant daily training stress leading up to the taper was optimized to maximize final performance for each taper length: allowing constant daily training stress to increase without bound does not result in unbounded performance gains. In this scenario, as taper length increases, larger training stress values are prescribed, as shown in FIG. 8(b). However, the fitness gains due to higher stresses and fatigue reduction due to a longer taper are balanced or outweighed by the increased fatigue due to higher stresses and fitness atrophy due to a longer taper. The result of this effect can be seen in the downturn of the dashed curve in FIG. 8(a).

Uniform Weekly Schedule Constraint

This section considers the case where an athlete might be constrained by their routine weekly schedule. In particular, we investigated the optimal training schedule when the daily training stresses were allowed to vary within the week, but every week was required to be identical. This creates a periodic training regimen with a seven day period. When relating this uniform weekly schedule to the uniform daily training stress case, the steady-state performance is of interest. The repeated variation within each week creates a periodic steady-state solution. shows the steady-state performance for the uniform weekly schedule that maximizes the average weekly performance in steady-state. These results demonstrate that the average performance value obtained using the uniform weekly schedule constraint is identical to the optimal performance value that was described above. This indicates that there may not be necessarily a unique solution that produces a maximum average performance when prescribing a uniform weekly training schedule constraint.

Maximum Daily Training Stress Constraint

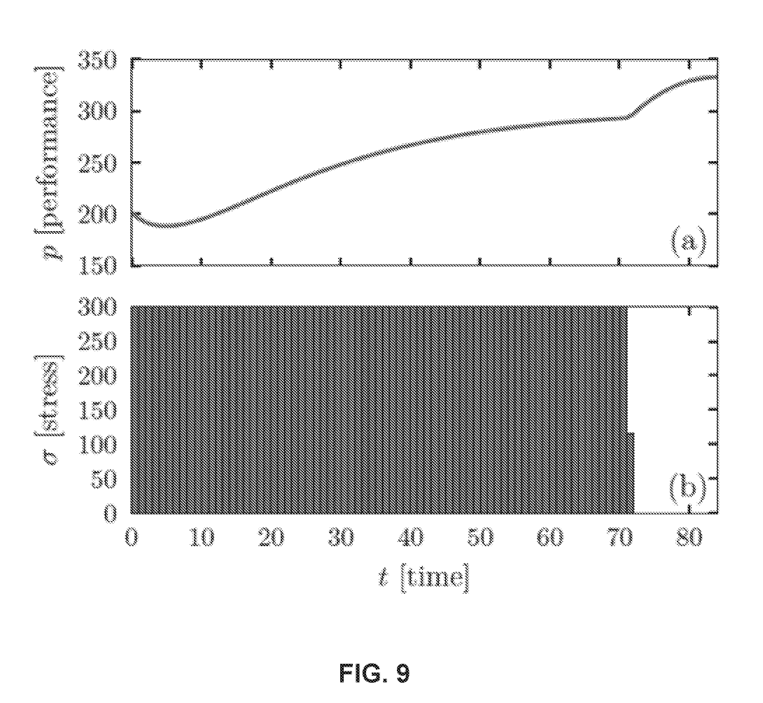

From a practical standpoint, an individual only has a limited number of hours to train each day. Thus, it is sensible to limit the training stress to a maximum allowable value that could reasonably be performed each day. For this purpose, we used a training stress of σ_{allow}=300, which roughly equates to 5-6 hours of training on a bicycle. FIG. 9 shows a training routine that results in optimal performance on race day, given a maximum training stress of σ_{allow}=300 as the only constraint. From these results, it is interesting to note that the optimal training routine does not contain a build-up in the daily training stress; thus, this constraint alone does not allow for a realistic progression in the training load as fitness improves. The optimal training routine requires training at the maximal allowable training stress every day until the taper begins. In order to develop more realistic training protocols, additional constraints must be considered.

Training Load Constraints

The constraints presented above demonstrate important aspects of the training-performance relationship, but they do not yield realistic training plans. This section considers the notion of a training load constraint, which results in optimal training plans that seek to balance performance gains with injury risk mitigation. The training plans that result from considering these training load constraints begin to resemble the training plans that coaches and traditional wisdom would recommend.

Maximum ATL Constraint

Acute training load (ATL) is commonly used by athletes to quantify the short-term effects of fatigue. This section seeks to optimize performance where daily training stress is constrained by both ATL and a maximum daily training stress. ATL can be defined as

Aₙ₊₁=Aₙ+(σₙ−Aₙ)(1−e^{-1/τ}) (6)

where, the acute training load Aₙ and the daily training stress σₙ on the n^{th} day were used along with the time constant τ=7 days to determine the acute training load Aₙ₊₁ on day n+1.

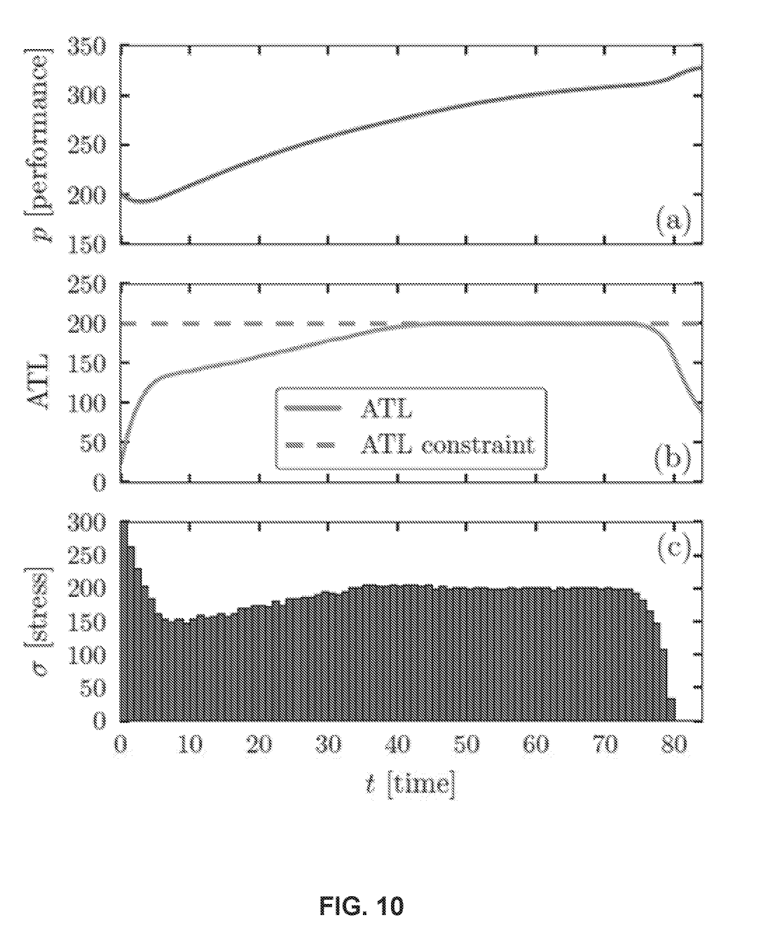

FIG. 10 depicts an example where the ATL was constrained to a maximum value of ATL_{allow}=200 and the daily training stress was constrained to a maximum value of σ_{allow}=300. It can be seen that the daily training stress is relatively high at the initial stages of training; therefore, the ATL constraint alone does not allow for a progressive build-up in the training load as fitness improves. However, after an initial transient period, the daily training stress is limited throughout the middle stages of training by the ATL constraint and begins to resemble a more realistic prescribed training strategy. Increasing the maximum ATL constraint increases final performance at a diminishing rate of return, but also increases the risk of fatigue-related injury.

Person-Specific Fatigue Constant

The ATL metric is a popular standardized metric, but it is not person-specific and is instead based on a generic time constant. An alternative approach is to use a person-specific constraint based on fatigue u from the nonlinear model. For example, the constraint explored in this section is defined as

σ_{allow}=300(0.1+0.9e^{-u/800}) (7)

This fatigue-based constraint allows for fairly high training stress when fatigue is low, but as fatigue increases, the allowable training stress asymptotically decreases.

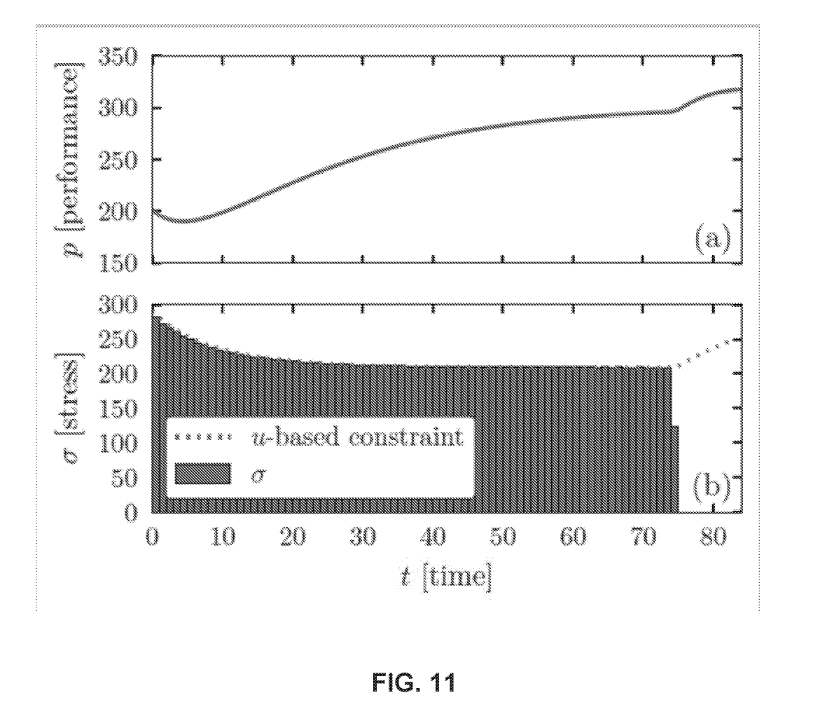

The resulting optimal training routine and performance are shown in FIG. 11. FIG. 11 is a plot showing (a) performance, and (b) training stress when a person-specific fatigue constraint and a daily maximum training stress of σ_{allow}=300 were applied.

Similar to the ATL constraint, the stresses are lower throughout the middle of the training program, but there is no progression in training load during the early stages of training. Therefore, these results indicate that an additional constraint is required to produce a progression in the allowable stresses during the earlier stages of training.

Maximum ATL/CTL Constraint

Chronic training load (CTL) is often calculated in conjunction with ATL to assess athletic fatigue. While ATL considers the short-term effects of fatigue, CTL considers the longer-term effects. This section considers a constraint that is based on the ratio of ATL/CTL. Conceptually, this constraint ensures that a longer period of training sufficiently prepares an individual for more intense short-term training effects. CTL can be calculated as

Cₙ₊₁=Cₙ+(σₙ−Cₙ)(1−e^{-1/τ}), (8)

where, the chronic training load Cₙ and the daily training stress σₙ on the n^{th} day were used along with the time constant i=42 days to determine the chronic training load Cₙ₊₁ on day n+1.

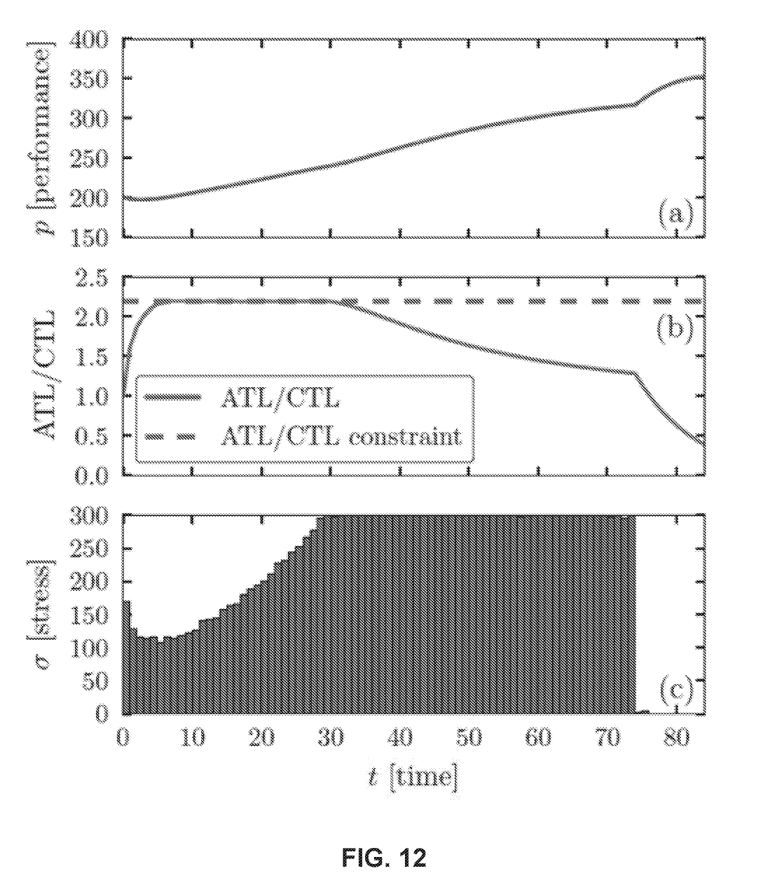

An optimal training routine that considers a maximum ATL/CTL constraint is depicted in FIG. 12. FIG. 12 is a plot showing (a) performance, (b) ATL/CTL ratio, and (c) daily training stress as optimized using the ATL/CTL constraint. A daily maximum stress of σ_{allow}=300 and (ATL/CTL)_{allow}=2.2 were imposed as constraints.

This constraint does a better job than previous constraints in terms of implementing a progression in training load during the early stages of the training program. However, as shown in this example, the training load during the first few days might still be relatively high; this was true for other ATL/CTL ratios that were also investigated. The training stress during the middle stages of the training program eventually becomes the maximal allowable daily value until the start of the taper.

Person-Specific Fatigue/Fitness Constraint

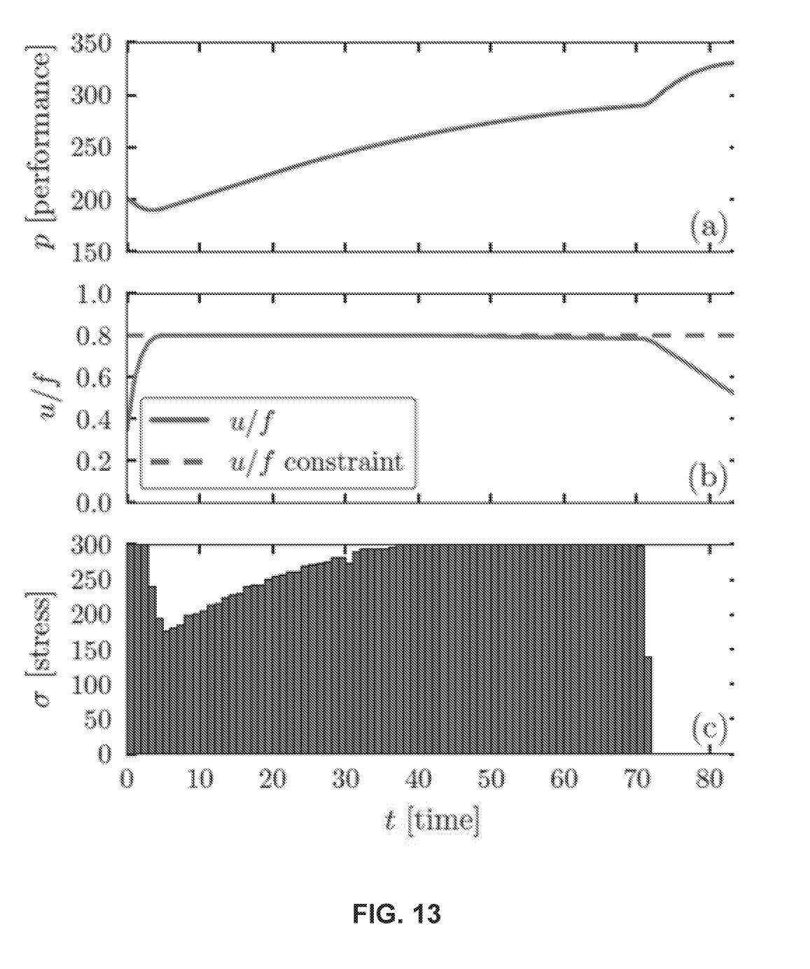

Analogous to the ATL/CTL constraint is a maximum fatigue/fitness constraint, which has the additional benefit of being person-specific. FIG. 13 shows that this fatigue/fitness constraint provides somewhat of a progression in the training stress at the onset of a training program. FIG. 13 is a plot showing (a) performance, (b) u/f, and (c) daily training stress as optimized using the u/f constraint. A daily maximum stress of σ_{allow}=300 and (u/f)_{allow}=0.8 were imposed as constraints. The behavior is similar to the case where the ATL/CTL ratio is used as a constraint. After a relatively large training stress during the first few days of training, the fatigue/fitness ratio forces progression in training stress until reaching the maximum stress of σ_{allow}=300, with a taper occurring at the end.

Training Progression Constraint

It is common for most training programs to progress the training load during the early stages of training. This strategy helps the individual improve their fitness before higher-stress loads are introduced and is often used to avoid injury. This section describes a constraint that improves upon using the ATL/CTL or the u/f ratio. More specifically, this section investigates constraining the training stress as a function of fitness. Intuitively, when fitness is low, the allowable stress should be low to limit the chance of injury, while as fitness increases, the allowable stress should approach a reasonable maximum value. An example of such a relationship is

σ_{allow}=300(1−0.9^{-f/150}) (9)

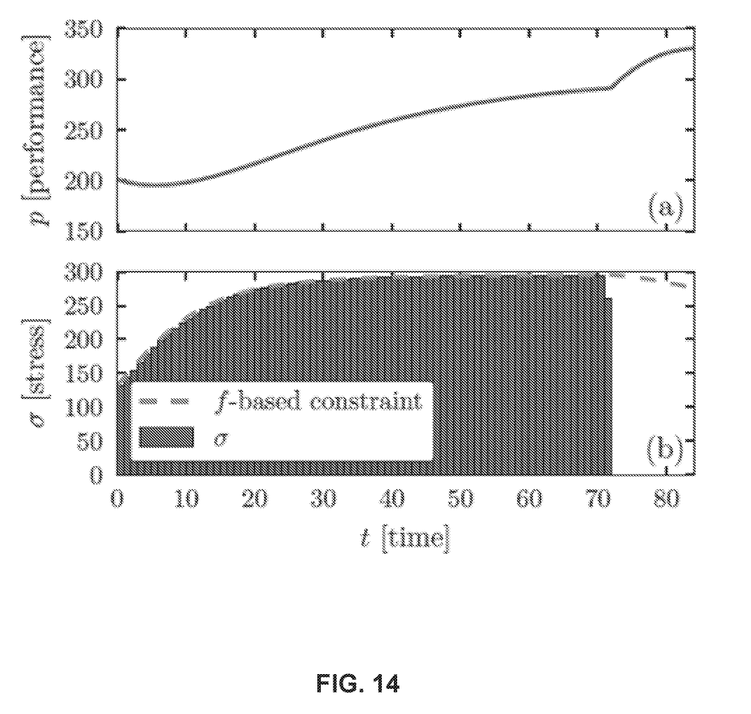

The resulting optimal training routine and performance shown in FIG. 14. FIG. 14 is a plot of (a) performance and (b) daily training stress when a person-specific f-based constraint and a daily maximum training stress of σ_{allow}=300 were applied. As shown this constraint provides a good build-up effect during the early stages of training.

Combination of Constraints

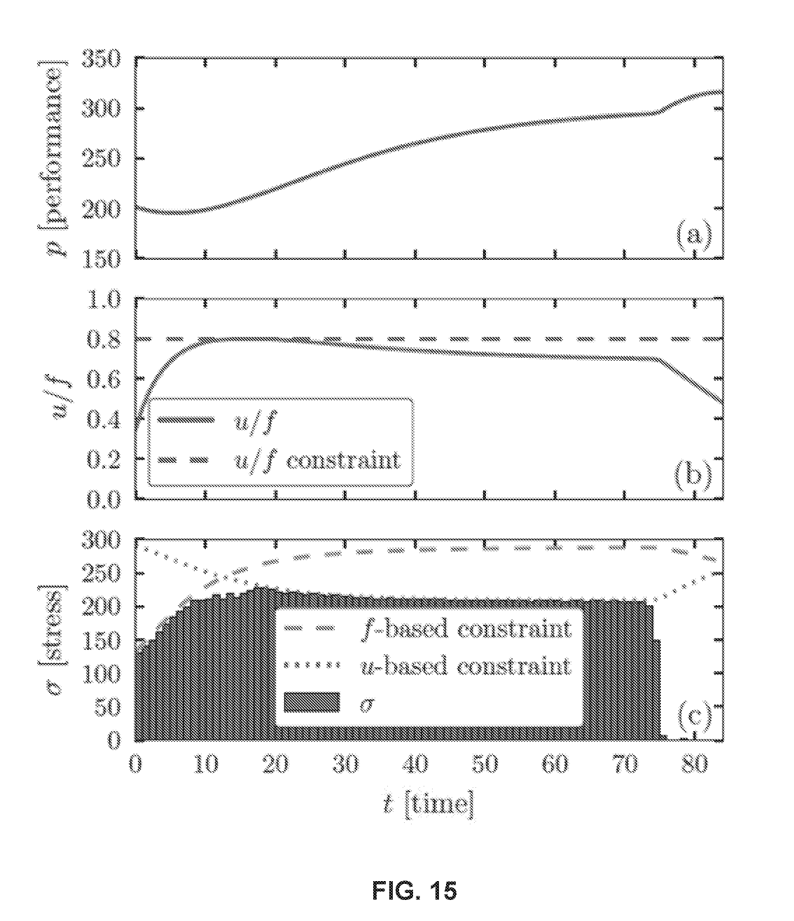

In order to develop more realistic training routines, a combination of constraints must be applied. For example, the fatigue-based maximum stress constraint, the maximum fatigue/fitness ratio constraint, and the fitness-based maximum stress constraint presented above were applied simultaneously, and the resulting training routine is shown in FIG. 15. This combination of constraints is much more similar to a training program that would be developed by a human coach—it has an initial period of training progression, mostly constant intensity for the middle of the season, and then a reasonable taper period. By incorporating both fitness-based and fatigue-based constraints, the routine attempts to avoid injury from effects of both low fitness and high fatigue. The fatigue/fitness ratio constraint helps to smooth the transition from the training progression at the beginning to the relatively constant intensity for the middle of the season. Out of all the constraint scenarios that were investigated in this study, this combination of constraints generates a more realistic training routine.

Human Performance Data

As shown above, the nonlinear model can be used to design optimal training programs subject to an individual's personal constraints. However, in order to apply the model in practice, the system parameters must first be estimated based on measurements of an individual's performance. This requires a parameter estimation algorithm, in this case a genetic algorithm, to optimize the parameters to fit the data. Parameter estimation was performed on a case study of historical data, and an assessment of the model limitations was performed.

Parameter Estimation

A retrospective study was conducted using historical data from one cyclist in order to assess the parameter estimation strategy and accuracy. In some embodiments, power meter pedals (for example, Garmin Vector power meter) were used to measure the power output produced by the cyclist (≤2% error). The performance was defined as the cyclist's average power output over the most intense 10-minute interval during each exercise bout. In some embodiments, the best fit was estimated using a genetic algorithm with the mean absolute error as the objective function.

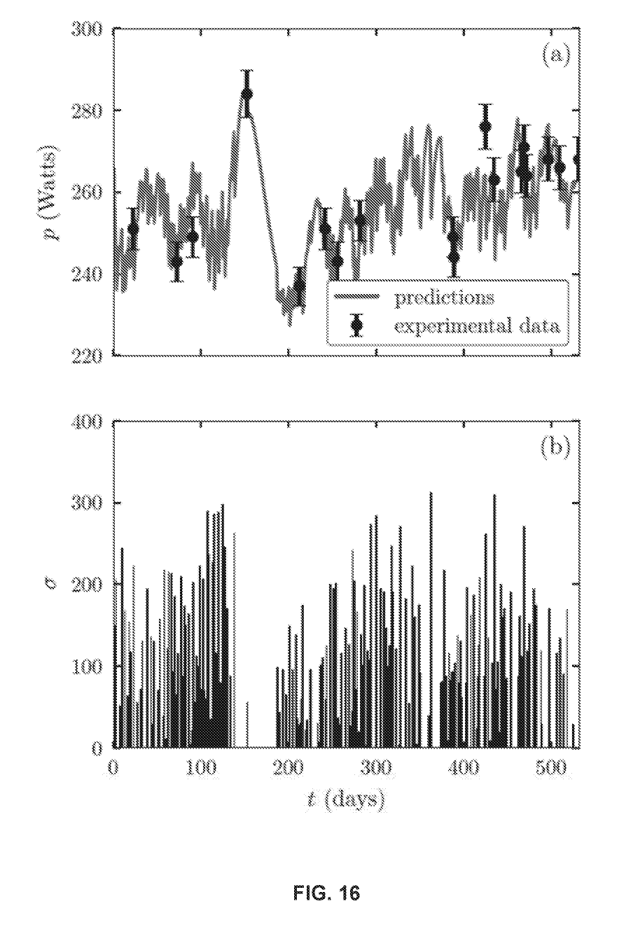

Since the analysis was performed on historical data, the cyclist was not necessarily exercising at maximum intensity for any one of the data points, so some performance measurements may have been below the true value. Unfortunately, the historical data included neither measurements of lactic acid and heart rate to quantify the effort level nor the individual's sleep and diet records to assess rest and recovery conditions. However, some knowledge of life events (for example illness) was available to eliminate data points that were not properly representative. The resulting data set is shown in FIG. 16.

Uncertainty Estimation

To determine the predictive limitations of the model, an assessment was performed based on cross-validation. This involved fitting the model to subsets of the data and then evaluating how well it applied to the remaining data. Of the 18 experimental trials that were conducted, 9 unique trials were randomly selected and the parameters were fit to those 9 points. This process of randomly selecting 9 unique trials and fitting the corresponding parameters was repeated 333 times in total. Using the parameters from the randomly selected trials and the initial conditions from the best fit to all trials, the model was integrated for the entire time period.

Results

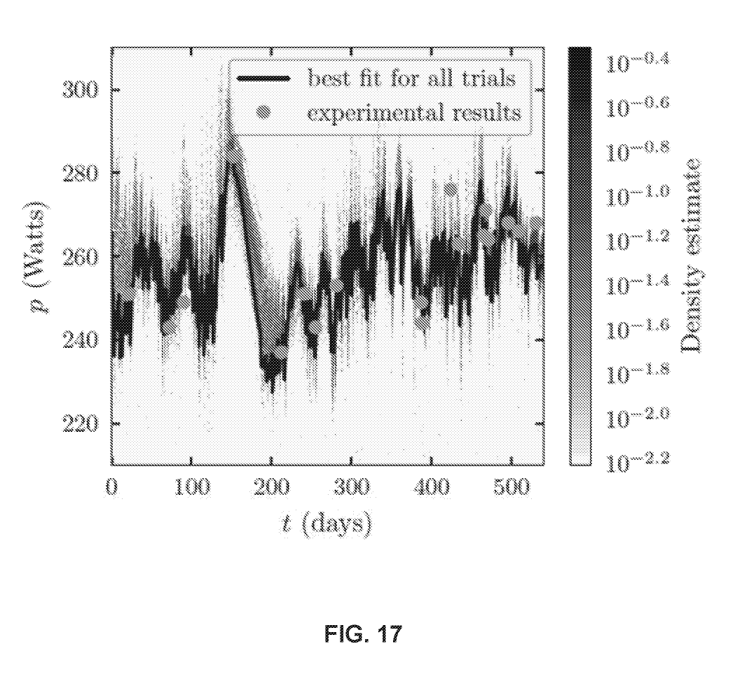

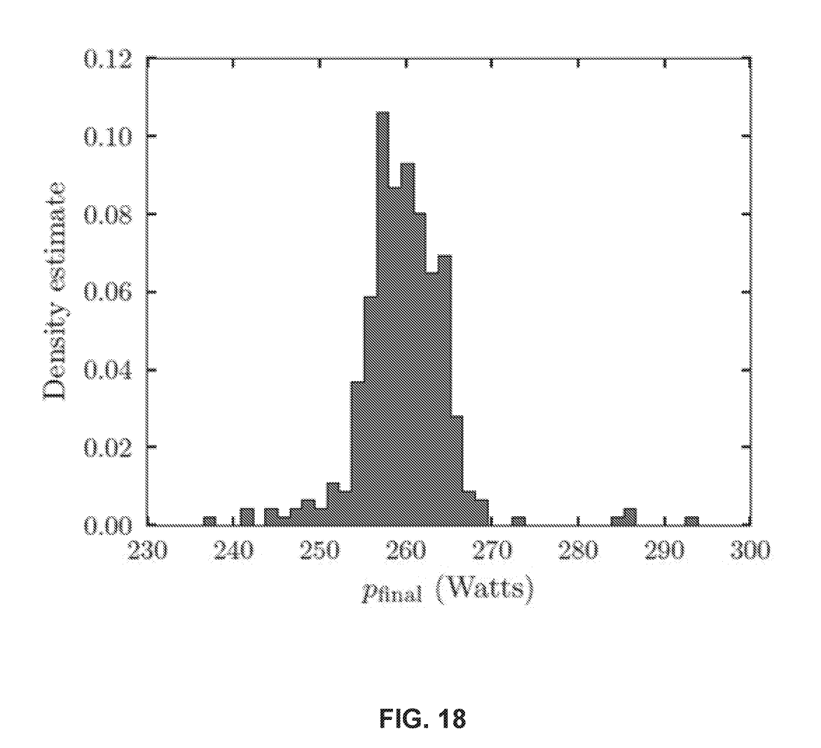

The results showed that the model fit the experimental data closely, and fitting to random subsets of data showed that some parameters had significantly more variation than others but the performance estimates were relatively consistent. The results of integrating the model using parameter values obtained from fitting the model parameters to all of the experimental data are shown in FIG. 16. The performance values predicted by the model matched the performance measurements closely, with a mean absolute error of 3.7 Watts. For comparison, this is less than the advertised measurement error from the power meter. FIG. 17 shows a normalized 2-D histogram of predicted performance using optimal parameters for randomly selected sets of trials. The counts were normalized such that the sum in each vertical slice is 1. Experimental data points are overlaid as dots. Parameters τ₁, τ₂, k₁, and k₂ had significantly more variation than the others. FIG. 17 depicts integration of the model using those parameter values. This 2-D histogram shows that there is a distribution in the performance predictions, but, except for a few regions, the variation is relatively small. FIG. 18 depicts a histogram of the predicted final performance values nine days after the last experimental data point. These estimates are approximately normally distributed with a reasonably small spread. FIG. 18 shows density estimation of final performance for randomly selected sets of trials. The vertical axis was normalized such that the total area under the histogram is 1.

Discussion

The results of fitting the model to the data in FIG. 16 are very good, given that the model makes predictions over a very long time period of 532 days.

There are a few possible causes of the errors between the performance predictions and the experimental data illustrated in FIG. 16. These include: 1) measurement errors; 2) the athlete exerting an inconsistent effort level; 3) variations in sleep and diet; and 4) limitations in the method of quantifying training stress. While the first two causes of error could easily be reduced by testing in a more controlled environment, the other two causes are phenomena that the model is not designed to capture. For example, variation in sleep and diet could affect the time constants for fitness gain and fatigue recovery, represented in the model as τ₁ and τ₂, respectively. Similarly, limitations in the stress metric could manifest as variations in the relative influence of stress on fitness and fatigue, i.e. k₁ and k₂. We hypothesize that variation in sleep and diet and limitations in the stress metric influenced the relatively large variations in τ₁, τ₂, k₁ and k₂.

The small variations seen in FIG. 17 and FIG. 18 show that even with some uncertainty in the parameters, fitting to randomly selected experimental trials can produce a relatively consistent final performance. Consistency in final performance predictions is an important criterion for competitive athletes.

Conclusions

The nonlinear model presented in Equation (1) and (2) successfully captures two essential effects, saturation and over-training, which are missed by linear models. As a result, the model can be used to optimize training routines specific to an individual's personal physiological characteristics, constraints, and performance goals.

Simulations for a representative set of parameter values suggest several useful conclusions. First, a taper is necessary to achieve maximum performance on race day since fitness decays at a slower rate than fatigue. Second, multiple solutions exist to achieve optimal average long-term performance, so if an individual is simply trying to maintain a regular schedule without a specific race day in mind, the individual can adjust their training schedule and maintain the same optimal performance. Third, different constraints provide various useful effects that alter the optimal daily training load. Fatigue-based constraints help limit stress during the middle of the training season but are not sufficient at the beginning if the starting fatigue is low. The ATL/CTL ratio and u/f ratio constraints provide a nice progression in training load except at the beginning when fatigue is low. They can also smooth the transition between fitness-based and fatigue-based constraints. A fitness-based constraint can provide a nice progression in training stress, starting from the first day. Finally, a combination of all of these constraints provides the most realistic training strategy that most closely matches conventional wisdom.

After applying the model to historical data and fitting parameters, the results matched the data quite well but also show areas for future research. The results suggest that there are variations in τ₁, τ₂, k₁, and k₂ that are not captured by the model; these could be explained by diet, sleep, and limitations in the stress metric, but additional modeling and experimentation are necessary.

It is important to note that the experimental work in this study defined training stress and performance using specific metrics related to cycling. However, the models, training constraints, and algorithms presented in this study can consider a large variety of training stress and performance metrics across a wide range of exercise modes including, but not limited to, running, cycling, swimming or any other type of athletic or physical activity.

The nonlinear model presented in this application captures important physiological effects missed by previous models, and this gives it new capabilities to design optimal training strategies specifically tailored to individuals' personal physiological characteristics, constraints, and performance goals.

The model is based on the physiological changes in response to exercise which manifest in terms of fitness and fatigue. Importantly, the model accounts for well-known physiological phenomena such as the concepts of saturation (diminishing returns) and over-training, which are not currently accounted for in other athletic performance models. Further, the model is also able to account for the personal physiology and fitness of an individual and does not rely on population-based statistical assumptions.

Parameter Estimation Algorithm

In some embodiments, the systems and methods provided herein utilize a heuristic parameter algorithm (for example, a genetic algorithm) to calibrate the model. Specifically, the genetic algorithm determines the person-specific constants that represent the physiological attributes of an individual (τ₁, τ₂, α, β, k₁, k₂, and p₀) and possibly the initial conditions f₀ and u₀. The genetic algorithm comprises a heuristic evolutionary algorithm that is based on the biological theory of evolution through natural selection. Genetic algorithms simulate a population of solutions over time and utilize evolutionary operators such as inheritance, selection, crossover, and mutation to arrive at an optimal solution. The genetic algorithm was configured to fit the predictive model parameters using input/output data (for example, inputs: training stress; outputs: performance). Since the parameter estimation algorithm is heuristic in nature, it can be run multiple times in order to generate a prediction with uncertainty bounds. Other types of parameter estimation algorithms could potentially be applied to this model as well to yield similar results.

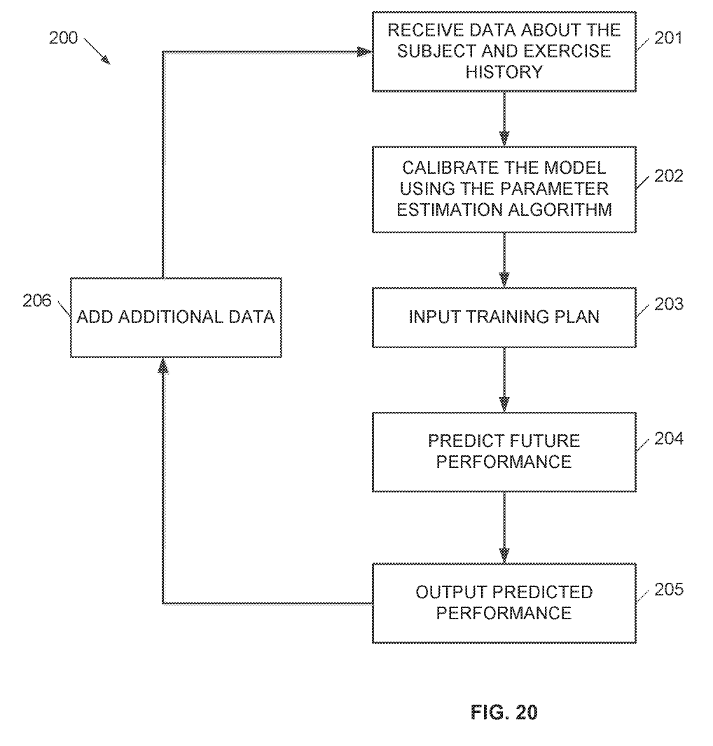

FIG. 20 is a flow chart of a method 200 for predicting performance for the subject, in accordance with some embodiments. In some embodiments, the method 200 at block 201 receives data about the subject and exercise history. The method 200 proceeds to block 202, wherein the method 200 calibrates the model described herein using the parameter estimation algorithm. The method 200 proceeds to block 203, wherein the method 200 inputs a training plan for the subject. The method 200 proceeds to block 204, wherein the method 200 predicts a future performance of the subject. The method 200 proceeds to block 205, wherein the method 200 outputs predicted performance of the subject. Finally, the method 200 proceeds to block 206, wherein the method 200 adds additional data associated with the subject to the existing data.

Optimal Training Algorithm

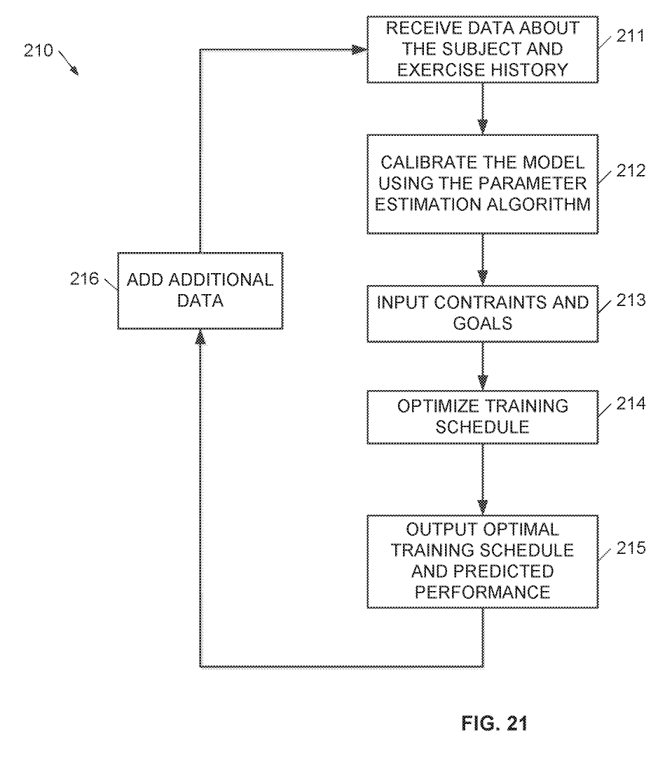

FIG. 21 is a flow chart of a method 210 for generating an improved or optimal training schedule and predicted performance for the subject, in accordance with some embodiments. In some embodiments, the method 210 at block 211 receives data about the subject and exercise history. The method 210 proceeds to block 212, wherein the method 210 calibrates the model using the parameter estimation algorithm. The method 210 proceeds to block 213, wherein the method 210 inputs constraints and goals associated with the subject and/or a training schedule or plan. The method 210 proceeds to block 214, wherein the method 210 optimizes the training schedule of the subject. The method 210 proceeds to block 215, wherein the method 210 outputs an optimal training schedule and predicted performance for the subject. Finally, the method 210 proceeds to block 216, wherein the method 210 adds additional exercise history data associated with the subject to the received biometric data of the subject.

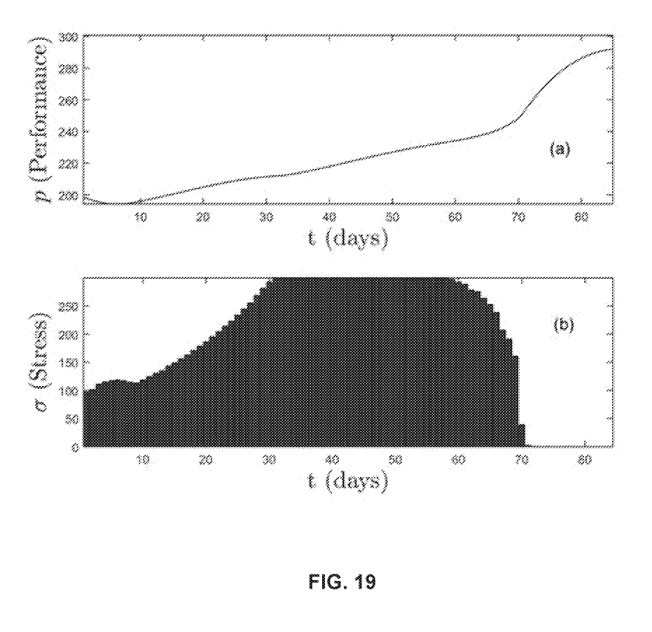

The systems and methods provided herein utilize a heuristic algorithm to design person-specific optimal training strategies. The algorithm is used to prescribe optimal exercise training to an individual based on their parameters which were determined during the parameter estimation process, their fitness goals, and their constraints. For some individuals, the athletic goal will be to maximize performance for an athletic competition, while for others it will simply be to reach and maintain a certain level of baseline fitness. The predictive model and algorithms are well suited to handle either of these scenarios. The constraints that an individual is subject to will vary considerably from person to person based on their health, fitness, and scheduling flexibility. The predictive model and algorithm are also well suited to handle this vast array of constraint possibilities. Scheduling constraints would restrict the days/hours that an individual could train based on their availability of time, while physiological constraints would be implemented to reduce the risk of developing both acute and chronic injuries. These physiological constraints might consist of daily/weekly stress limits, as well as limits associated with acute and chronic training loads. Other metrics could also be used as constraints as well. These algorithms particularly excel at generating optimal “tapering” strategies to minimize fatigue and maximize performance before a competitive athletic event. An example of an optimal training routine is shown in FIG. 19.

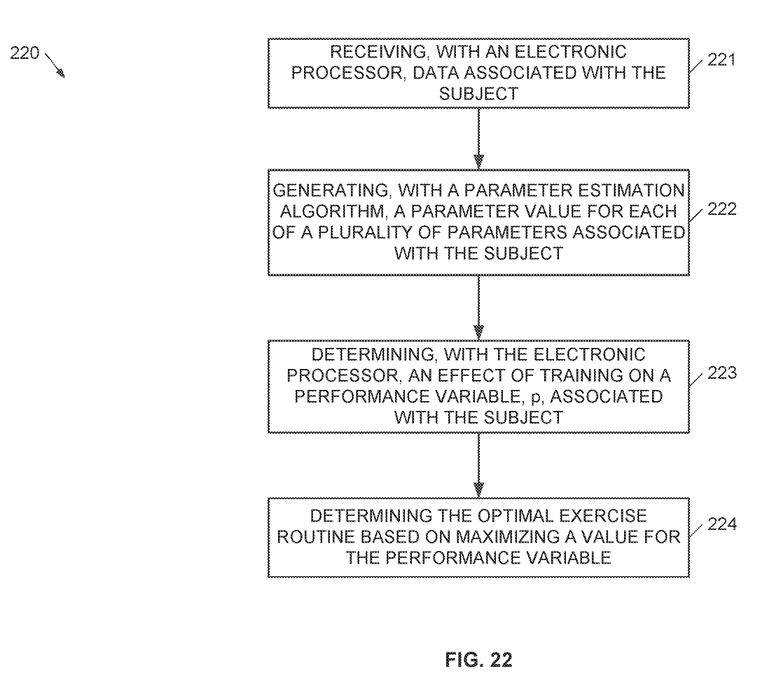

FIG. 22 is a flow chart of a method 220 determining an optimal exercise routine for the subject, in accordance with some embodiments. In some embodiments, the method 220 includes, at block 221, receiving, with the electronic processor 102, data associated with the subject. In some embodiments, the data associated with the subject is one or more of the following: (i) physiological attribute data of the subject, (ii) calibration data consisting of measured training, (iii) calibration data consisting of performance data from past exercise, (iv) training stress data, (v) fitness data, (vi) fatigue data, (vii) desired constraints data, and (viii) desired goals data. The method 220 proceeds to block 222, wherein the method 220 includes generating, with a parameter estimation algorithm, a parameter value for each of a plurality of parameters associated with the subject. In some embodiments, the parameter estimation algorithm includes a heuristic algorithm. For example, the heuristic algorithm can be at least one of a genetic algorithm, simulated annealing algorithm, and particle swarm algorithm. The method 220 further proceeds to block 223, wherein the method 220 includes determining, with the electronic processor 102, an effect of training on a performance variable, p, associated with the subject. The method 220 further proceeds to block 224, wherein the method 220 includes determining the optimal exercise routine based on maximizing a value for the performance variable.

In other embodiments, the system may further include one or more performance measurement devices. Such devices may be used, for example, to measure the subject's test performance. Examples include, but are not limited to, a global positioning system (“GPS”) receiver, a heart rate monitor, a stopwatch, a power meter, and the like. In some embodiments, the performance measurement device may include a processing unit capable of obtaining the training metric. Accordingly, such data may be directly uploaded to the computing device via accessing a drive (USB, CD) of the computing device or via direct communication link (infrared, cable, wireless Internet).

The systems provided herein may be used to perform one or more steps of the methods of the present disclosure. For example, the subject may enter the performance goal information or any other information using a keyboard, mouse, touch-screen, or any other device. Similarly, the user may also enter commands relating to the computation of performance models, etc.

The systems and methods provided herein have several significant advantages when compared to current methods for modeling and predicting athletic performance. Traditional models have sought to model and predict athletic performance based on linear assumptions and therefore fail to account for well-known physiological phenomena such as the concepts of saturation (diminishing returns) and over-training. Furthermore, the traditional models and methods are capable of fitting experimental data to some extent, but are limited in terms of their practical applications, specifically with regards to predicting future performance based on training and the development of optimal training strategies. The predictive models described herein incorporate nonlinear aspects of human physiology, which allows the predictive model to represent the dynamics of fitness adaptations and the onset of fatigue with a significantly more sophisticated approach. The predictive models described herein allows for the application of optimization algorithms to physiological data that enable not only the prediction of athletic performance, but also the design of optimal training strategies for athletes based on their personal physiology, fitness, athletic/rehabilitation goals, and constraints.

The systems and methods provided herein, and the predictive models incorporated therein, have significant applications to athletics, health, and fitness. The ability to predict athletic performance based on training inputs offers a tremendous advantage to subjects participating in competitive athletics. The systems and methods disclosed herein enable subjects to design optimal training strategies for athletes based on their personal physiology, fitness, athletic/rehabilitation goals, and constraints using both analytical and computational methods. In addition to providing a framework of optimal training, the systems and methods also provide for optimal tapering strategies, suggesting how much time a subject should rest before a competitive event to be free from fatigue. The systems and methods will be useful to subjects training for athletic competitions and also for subjects rehabilitating from injuries, as the optimization algorithm can be easily configured for either scenario. These systems and methods described herein are not limited to those subjects who are athletes, but also have applicability to those subjects who exercise to maintain their health and fitness. The systems and methods described herein can be used to help these subjects attain their fitness goals in a safe and efficient manner. Yet another benefit of the present disclosure is that it has immediate applications for the health and fitness communities and is easily implementable with existing physiological data collection technologies.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein is presently representative of preferred embodiments, is exemplary, and is not intended as limitations on the scope of the invention. Changes therein and other uses will occur to those skilled in the art which are encompassed within the spirit of the invention as defined by the scope of the claims.

What is claimed is:

1. A method for determining an exercise routine of a subject, the method comprising:
receiving, with an electronic processor, data associated with the subject;
generating, with a parameter estimation algorithm, a parameter value for each of a plurality of parameters associated with the subject;
determining, with the electronic processor, an effect of training on a performance variable, p, associated with the subject;
determining the exercise routine based on maximizing a value for the performance variable;
wherein the performance variable is a function of a fitness variable of the subject and a fatigue variable of the subject;
wherein the fitness variable and a time derivative of the fitness variable have a nonlinear relationship; and
wherein the fatigue variable and a time derivative of the fatigue variable have a nonlinear relationship.

2. The method of claim 1, wherein the data associated with the subject is selected from the group consisting of: (i) physiological attribute data of the subject, (ii) calibration data consisting of measured training, (iii) calibration data consisting of performance data from past exercise, (iv) training stress data, (v) fitness data, (vi) fatigue data, (vii) desired constraints data, and (viii) desired goals data.

3. The method of claim 1, wherein the relationships between the performance variable, the fitness variable, and the fatigue variables are expressed as:
$$p = {p}_{o} \pm ( f - u )$$
$$\dot{f} + \frac{1}{{τ}_{1}} {f}^{α} = {k}_{1} σ ( t )$$
$$\dot{u} + \frac{1}{{τ}_{2}} {u}^{β} = {k}_{2} σ ( t )$$
wherein σ(t) is a training stress impulse variable,
τ₁, τ₂, α, β, k₁, and k₂ are subject-specific parameters representing physiological attributes of the subject,
p₀ is a baseline performance parameter of the subject in an untrained state,
f is a fitness variable of the subject,
\dot{f} is a time derivative of the fitness variable,
u is a fatigue variable of the subject,
\dot{u} is a time derivative of the fatigue variable, and
p is the performance variable associated with the subject.

4. The method of claim 1, further comprising:
determining a predicted performance of the subject based on optimizing the performance variable.

5. The method of claim 1, wherein the parameter estimation algorithm comprises a heuristic algorithm selected from the group consisting of a genetic algorithm, simulated annealing algorithm, and particle swarm algorithm.

6. The method of claim 1, wherein maximizing the value for the performance variable includes using a heuristic algorithm selected from the group consisting of a genetic algorithm, simulated annealing algorithm and particle swarm algorithm.

7. The method of claim 1, wherein the exercise routine of the subject is associated with an individual sport.

8. The method of claim 7, wherein the individual sport is selected from the group consisting of swimming, running, cycling, rowing, strength training and hammer throwing.

9. A system for determining an exercise routine for a subject, the system comprising:
a sensor to generate data associated with the subject; and
a computing device including an electronic processor configured to
receive data associated with the subject,
generate, using a parameter estimation algorithm, a parameter value for each of a plurality of parameters associated with the subject,
determine an effect of training on a performance variable, p, associated with the subject,
determine the optimal exercise routine based on optimizing the performance variable,
wherein the performance variable is a function of a fitness variable of the subject and a fatigue variable of the subject,
wherein the fitness variable and a time derivative of the fitness variable have a nonlinear relationship, and
wherein the fatigue variable and a time derivative of the fatigue variable have a nonlinear relationship.

10. The system of claim 9, wherein the relationships between the performance variable, the fitness variable, and the fatigue variables are expressed as:
$$p = {p}_{o} \pm ( f - u )$$
$$\dot{f} + \frac{1}{{τ}_{1}} {f}^{α} = {k}_{1} σ ( t )$$
$$\dot{u} + \frac{1}{{τ}_{2}} {u}^{β} = {k}_{2} σ ( t )$$
wherein σ(t) is a training stress impulse variable,
τ₁, τ₂, α, β, k₁, and k₂ are subject-specific parameters representing physiological attributes of the subject,
p₀ is a baseline performance parameter of the subject in an untrained state,
f is a fitness variable of the subject,
\dot{f} is a time derivative of the fitness variable,
u is a fatigue variable of the subject,
\dot{u} is a time derivative of the fatigue variable, and
p is the performance variable associated with the subject.

11. The system of claim 9, wherein the sensor includes at least one selected from the group consisting of a biometric sensor and an environmental sensor.

12. The system of claim 9, wherein the sensor is selected from the group consisting of a heart rate monitor, an oxygen uptake (VO₂) sensor, a power meter, a GPS system, a timing device, an inertial measurement unit (IMU), an accelerometer, a gyroscope, a magnetometer, a step sensor, a position sensor, a force sensor, a velocity sensor, a torque sensor, a cadence sensor, an oxygen saturation (SmO₂) sensor, and a blood lactate (BLa) sensor.

13. The system of claim 9, wherein the computing device is a portable communication device.

14. The system of claim 9, wherein the portable communication device includes at least one selected from the group consisting of a smart phone, a wearable health-monitoring device, a smart watch, and smart glasses.

15. A non-transitory computer-readable medium containing computer-executable instructions that when executed by one or more electronic processors cause the one or more electronic processors to:
receive data associated with the subject;
generate a parameter value for each of a plurality of parameters associated the subject using a heuristic parameter estimation algorithm;
determine an effect of training on a performance variable, p, associated with the subject;
determine the optimal exercise routine based on optimizing the performance variable;
wherein the performance variable is a function of a fitness variable of the subject and a fatigue variable of the subject,
wherein the fitness variable and a time derivative of the fitness variable have a nonlinear relationship, and
wherein the fatigue variable and a time derivative of the fatigue variable have a nonlinear relationship.

16. The non-transitory computer-readable medium of claim 15, further comprising computer-executable instructions that when executed by one or more electronic processors cause the one or more electronic processors to
determine the optimal exercise routine based on maximizing a value for the performance variable, wherein the relationships between the performance variable, the fitness variable, and the fatigue variables are expressed as:
$$p = {p}_{o} \pm ( f - u )$$
$$\dot{f} + \frac{1}{{τ}_{1}} {f}^{α} = {k}_{1} σ ( t )$$
$$\dot{u} + \frac{1}{{τ}_{2}} {u}^{β} = {k}_{2} σ ( t )$$
wherein σ(t) is a training stress impulse variable, τ₁, τ₂, α, β, k₁, and k₂ are subject-specific parameters representing physiological attributes of the subject, p₀ is a baseline performance parameter of the subject in an untrained state, f is a fitness variable of the subject, \dot{f} is a time derivative of the fitness variable, u is a fatigue variable of the subject, \dot{u} is a time derivative of the fatigue variable, and p is the performance variable associated with the subject.

17. The non-transitory computer-readable medium of claim 15, further comprising computer-executable instructions that when executed by one or more electronic processors cause the one or more electronic processors to
receive information associated with the subject, wherein the information is selected from the group consisting of (i) physiological attributes of the subject, (ii) calibration data consisting of measured training, (iii) calibration data consisting of performance data from past exercise, (iv) training stress data, (v) fitness data, and (vi) fatigue data.

18. The non-transitory computer-readable medium of claim 16, wherein the parameter estimation algorithm comprises a heuristic algorithm selected from the group consisting of a genetic algorithm, a simulated annealing algorithm, and a particle swarm algorithm.
